(19) United States
(12) Patent Application Publication
(10) Pub. No.: US 2025/0034195 A1
(43) Pub. Date: Jan. 30, 2025

(54) METHOD FOR THE PRODUCTION OF SPHINGOLIPIDS

(71) Applicant: CarboCode S.A., Coimbra (PT)

(72) Inventor: Halina ZHYLITSKAYA, Coimbra (PT)

(21) Appl. No.: 18/716,083

(22) PCT Filed: Nov. 29, 2022
(86) PCT No.: PCT/EP2022/083675

§ 371 Date: Jun. 3, 2024

(30) Foreign Application Priority Data
Dec. 2, 2021 (PT) 117615
Dec. 2, 2021 (PT) 117616
Dec. 15, 2021 (PT) 117639

(51) Int. Cl. C07H 15/04; C07D 251/30; C07H 1/00

(52) U.S. Cl. CPC C07H 15/04; C07D 251/30; C07H 1/00

(57) ABSTRACT

The present invention relates to a novel and efficient method for the production of sphingolipids via the N-acylation of lysosphingolipids. The method comprises the use of 1,3,5-triazine-based acylating agents, and is especially suitable for the production of ceramides and glycosphingolipids. The present invention further relates to novel 1,3,5-triazine-based acylating agents.

### FILED OF THE INVENTION

The present invention relates to a novel and efficient method for the production of sphingolipids via the N-acylation of lysosphingolipids such as sphingoid bases and glycosylated sphingoid bases. The method is based on the use of 1,3,5-triazine-based acylating agents, and is especially suitable for the production of ceramides and glycosphingolipids.

### BACKGROUND

Sphingolipids are an important class of polar lipids mainly found on the surface of eukaryotic cells. Sphingolipids are structurally characterized by a sphingoid base backbone and can be divided in different classes such as, ceramides, and glycosphingolipids.

Ceramides are N-acylated sphingoid bases lacking additional head groups at the 1-position of the sphingoid base backbone, and wherein the N-acyl group of ceramides typically derives from a fatty acid. Glycosphingolipids (GSLs) are glycoconjugates deriving from ceramides, wherein a glycan moiety is linked to the 1-hydroxyl group of a ceramide via a glycosidic linkage.

Sphingolipids are involved in diverse biological processes and play important structural and functional roles such as cell-cell recognition, communication, and intercellular adhesion. Particularly, GSLs such as gangliosides are found in the brain and play roles in neurological diseases (Kolter, ISRN Biochem. 2012), whereas ceramides are the main constituent of the stratum corneum lipid layer and have a major role in the water-retaining properties of the epidermis, as well as in the barrier function of the skin.

Accordingly, sphingolipids hold great potential as therapeutics, cosmetics, and as tools for the study of important biological processes. However, they are not readily available for fundamental and clinical research. In fact, sphingolipids such as ceramides and GSLs are characterized by a high structural complexity and their preparation represents a challenge.

Current processes for the preparation of sphingolipids are typically based on extraction from animal brains, or animal epidermal tissues (EP 3095451 A1, U.S. Pat. No. 5,532,141 A). However, extraction and isolation of sphingolipids from animal sources is a laborious and costly process, and typically yields the desired compounds in low amounts and with low purities. Furthermore, the obtained sphingolipids may be potentially unsafe due to the presence of hazardous biological contaminants.

Alternatively, sphingolipids such as ceramides and GSLs may be obtained via the N-acylation of lysosphingolipids.

Typically, lysosphingolipids are defined as sphingolipid breakdown products which lack the amide-linked fatty acyl group at the 2-position of the sphingoid base backbone. Accordingly, for each parental sphingolipid there is a corresponding lysosphingolipid that has an identical head group at the 1-position but lacks the amide-bound fatty acyl group at the 2-position (Hannun et al., Science 1989, 243, 500-507).

Attempts have been made for the N-acylation of lysosphingolipids via chemical or enzymatic approaches.

Current chemical N-acylation methods are typically based on the use of standard amide bond coupling reagents such as N-hydroxysuccinimide (NHS), 1,3-dicyclohexylcarbodiimide (DCC), or 1-ethyl-3-(3-dimethylaminopropyl)carbodiimide (EDC) (Skolova et al, Biochim Biophys Acta Biomembr. 2017, 1859, 824-834, WO0172701 A1). Disadvantages connected to these methods include the use of expensive and unstable coupling reagents, which typically require the use of water-free solvents and/or the reaction must be performed under exclusion of air, thus rendering the scale up difficult.

Current enzymatic approaches are based on the use of lipases (WO1994026919A1) which, however, are typically not specific for the amino group of a lysosphingolipid but may also act on hydroxyl groups, thus leading to the formation of mixtures of N- and O-acylation products, and thus requiring a lengthily and costly purification of the target compound.

Furthermore, current chemical and enzymatic approaches for the N-acylation of lysosphingolipids mainly rely on the use of lysosphingolipids obtained from animal sources.

Accordingly, there is a demand for the development of novel and improved methodologies which enable the efficient and large-scale N-acylation of lysosphingolipids characterized by high technological feasibilities and low costs.

### SUMMARY OF THE INVENTION

In a first aspect, the present invention relates to a method for the production of a sphingolipid of formula (1):

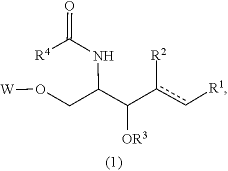

wherein
W is hydrogen or a glycosyl moiety,
R¹ is H, aryl, or a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of a hydroxyl group, an alkoxy group, an acyloxy group, an acylamido group, a thiol, a thioether or a phosphorus-containing functional group,
R² is H or —OR⁵, wherein R⁵ is selected from hydrogen, a substituted or unsubstituted C₁₋₆ alkyl, or a substituted or unsubstituted C₁₋₆ acyl,
the bond [symbol] may be a double or a single bond when R² is H, or is a single bond when R² is —OR⁵,
R³ is H, a substituted or unsubstituted C₁₋₆ alkyl, or a substituted or unsubstituted C₁₋₆ acyl,
R⁴ is selected from hydrogen, a substituted or unsubstituted aryl, a heteroalkyl, a substituted or unsubstituted C₁₋₃₁ alkyl, preferably a substituted or unsubstituted C₉₋₃₁ alkyl,
the method comprising:
reacting a lysosphingolipid of formula (2):

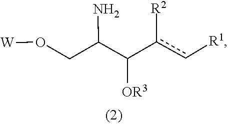

wherein W, the bond [symbol] R¹, R² and R³ are as defined as for the sphingolipid of formula (1), with one or more triazine-based acylating agents of formula (3):

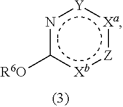

wherein

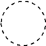

represents a conjugated system of bonds such that either two or three double bonds are present in the ring,
Y is selected from C(O—C(═O)R⁴), or C(═O),
X^{a} is selected from N, NR⁶, or N(C(═O)R⁴)
X^{b} is selected from N, or NR⁶
Z is selected from C(═O), or C(OR⁶),
and provided that:
when Y is C(O—C(═O)R⁴), Z is C(OR⁶), X^{a} and X^{b} are N, and three double bonds are present in the ring, or
when Y is C(O—C(═O)R⁴), Z is C(═O), one of X^{a} and X^{b} is N and the other group is NR⁶, and two double bonds are present in the ring, or
when Y is C(═O), Z is C(OR⁶), X^{a} is N(C(═O)R⁴), X^{b} is N, and two double bonds are present in the ring,
and wherein
R⁴ is as defined as for the sphingolipid of formula (1),
R⁶ is selected from methyl, ethyl, 2,2,2-trifluoroethyl, and substituted or unsubstituted benzyl,
thereby producing the sphingolipid of formula (1).

In a second aspect, the present invention relates to a method for the production of a sphingolipid of formula (1), wherein the method further comprising steps of producing the triazine-based acylating agent formula (3), or the combination thereof.

In a third aspect, the present invention relates to method for the production of a sphingolipid of formula (1), wherein the triazine-based acylating agent of formula (3), or the combination thereof, is produced via the steps of:
reacting a carboxylic acid of formula (16):

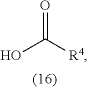

wherein
R⁴ is as defined as for the sphingolipid of formula (1), with a compound of formula (17):

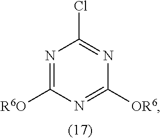

wherein R⁶ is as defined as for the triazine-based acylating agent of formula (3), or the combination thereof,
in the presence of an organic base, thereby producing the triazine-based acylating agents of formula (3), or the combination thereof;
isolating the triazine-based acylating agent of formula (3), or the combination thereof, via precipitation;
and wherein the steps of producing the triazine-based acylating agent of formula (3), or the combination thereof, are performed in a non-halogenated solvent.

In a fourth aspect, the present invention relates to a triazine based acylating agent of formula (3), or a combination thereof:

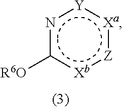

wherein

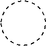

represents a conjugated system of bonds such that either two or three double bonds are present in the ring;
Y is selected from C(O—C(═O)R⁴), or C(═O);
X^{a} is selected from N, NR⁶, or N(C(═O)R⁴);
X^{b} is selected from N, or NR⁶;
Z is selected from C(═O), or C(OR⁶);
provided that:
when Y is C(O—C(═O)R⁴), Z is C(OR⁶), X^{a} and X^{b} are N, and three double bonds are present in the ring, or
when Y is C(O—C(═O)R⁴), Z is C(═O), one of X^{a} and X^{b} is N and the other group is NR⁶, and two double bonds are present in the ring, or
when Y is C(═O), Z is C(OR⁶), X^{a} is N(C(═O)R⁴), X^{b} is N, and two double bonds are present in the ring,
and wherein
R⁶ is selected from methyl, ethyl, 2,2,2-trifluoroethyl, and substituted or unsubstituted benzyl,
R⁴ is a substituted or unsubstituted C₉₋₃₁ alkyl selected from the group consisting of substituted or unsubstituted C₉₋₃₁ alkyl of formula (8) and (9):

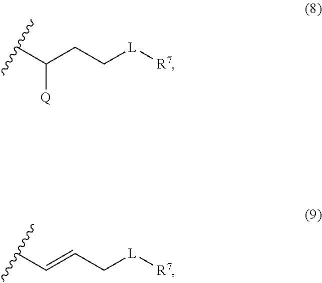

wherein
Q is selected from —H, —OH, —Cl, —Br, or —I,
L is a straight-chain C₆₋₂₈ alkylene, which may be saturated or contain one or more double bonds and or triple bonds,
R⁷ is hydrogen, or —OR⁸, wherein R⁸ is hydrogen or a straight-chain C₂₋₃₀ acyl which may be saturated or contain one or more double bonds;
and provided that:
when R⁶ is a methyl and Q and R⁷ are hydrogen, L of the alkyl group of formula (8) is not an unsaturated straight-chain C₁₄ alkylene, or L of the alkyl group of formula (8) is not a straight-chain C₁₅ alkylene, or L of the alkyl group of formula (8) is not a straight-chain C₁₈ alkylene.

In a fifth aspect, the present invention relates to a combination comprising triazine base acylating agents of formula (10)-(13):

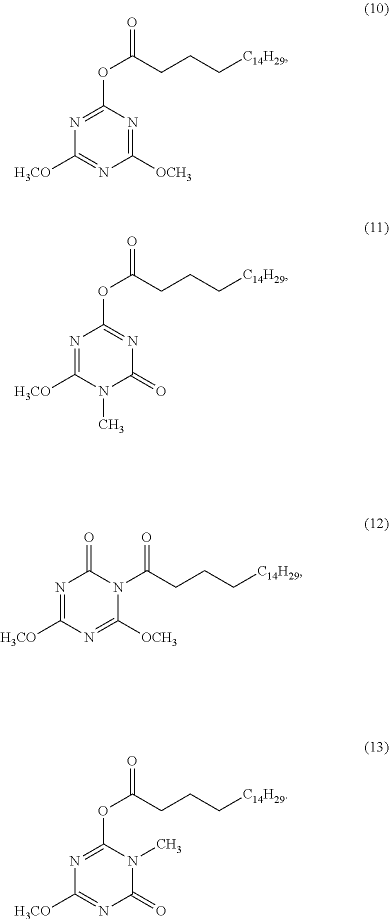

and wherein each of said compounds is present, in said combination, in the amount from about 1% to about 99%.

### DETAILED DESCRIPTION OF INVENTION

The present inventors have established for the first time an efficient and economic method for the production of sphingolipids via the chemical N-acylation of lysosphingolipids, and wherein the lysosphingolipids used as the starting materials are preferably obtained via synthetic and/or biotechnological approaches.

Surprisingly, the present inventors have found that lysosphingolipids can be N-acylated using a robust and easily accessible 1,3,5-triazine-based acylating agent of formula (3), or a combination thereof:

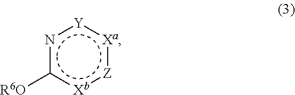

wherein

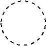

represents a conjugated system of bonds such that either two or three double bonds are present in the ring;
Y is selected from C(O—C(═O)R⁴), or C(═O);
X^{a} is selected from N, NR⁶, or N(C(═O)R⁴);
X^{b} is selected from N, or NR⁶;
Z is selected from C(═O), or C(OR⁶);
and provided that:
when Y is C(O—C(═O)R⁴), Z is C(OR⁶), X^{a} and X^{b} are N, and three double bonds are present in the ring, or
when Y is C(O—C(═O)R⁴), Z is C(═O), one of X^{a} and X^{b} is N and the other group is NR⁶, and two double bonds are present in the ring, or
when Y is C(═O), Z is C(OR⁶), X^{a} is N(C(═O)R⁴), X^{b} is N, and two double bonds are present in the ring;
and wherein
R⁴ is as defined as for the sphingolipid of formula (1),
R⁶ is selected from methyl, ethyl, 2,2,2-trifluoroethyl, and substituted or unsubstituted benzyl.

The process described in the present invention gives access to a diverse set of biologically relevant sphingolipids in high purity and yields. Furthermore, the process does not require the use of water-free solvents and/or exclusion of air, and the produced sphingolipids can be isolated directly from the reaction mixture via precipitation in high purity and yields.

Non-limiting embodiments of different aspects of the invention are described below and illustrated by non-limiting examples.

The terms, definitions and embodiments described throughout the specification of the invention relate to all aspects and embodiments of the invention.

The term “a” grammatically is a singular, but it may as well mean the plural of e.g., the intended compound. For example, a skilled person would understand that in the expression “a lysosphingolipid of formula (2)”, the provision of not only one single a lysosphingolipid of formula (2), but of a variety of lysosphingolipids of the same type is meant.

In formulas representing a moiety, or a group such as for example the alkyl moiety of formula (8) or (9), or the acyl groups of formula (20)-(22), or (23)-(31) the symbol [symbol] means a point of attachment to another group or atom.

As used herein, the various functional groups or substituents represented will be understood to have a point of attachment at the functional group or atom having the dash (-). For example, in the case of —C(═O) it will be understood that the point of attachment is the carbon atom. If a group is listed without a dash, then the attachment point is indicated by the plain and ordinary meaning of the recited group.

As used herein the letters N, C, O and H refer to a nitrogen atom, to a carbon atom, to an oxygen atom and to a hydrogen atom, respectively.

The skilled person would understand that when speaking of position C-1, C-2, C-3, C-4, C-5 etc., reference is herein always made to the respective carbon atoms of sphingolipids of formula (1), (32), or (33), or lysosphingolipids of formula (2), (14), or (15).

The expression “one of X^{a} and X^{b} is N and the other group is NR⁶,” means that when X^{a} is N the other group X^{b} is NR⁶, or when X^{b} is N the other group X^{a} is NR⁶.

As used herein, the term “alkyl” refers to an acyclic straight or branched hydrocarbyl radical having 1-50 carbon atoms which may be saturated or contain one or more double and/or triple bonds (so, forming for example an alkenyl or an alkynyl), and/or which may be substituted or unsubstituted, as herein further described. Examples of “alkyl” include, but are not limited to, methyl, ethyl, n-propyl, isopropyl, isobutyl, n-butyl, sec-butyl, tert-butyl, isopentyl, n-pentyl, neo-pentyl, n-hexyl, ethenyl, propenyl, 1-butenyl, 2-butenyl, isobutenyl, 1-pentenyl, 2-pentenyl, 2-methyl-1-butenyl, 3-methyl-1-butenyl, 2-methyl-2-butenyl, 1-hexenyl, 2-hexenyl, 3-hexenyl, methylpentenyl, dimethylbutenyl, ethynyl, propynyl, 1-butynyl, 2-butynyl, pentynyl, and hexynyl, each of which may be substituted or unsubstituted. Typically, the term alkyl refers to a straight acyclic hydrocarbyl group having 1-32 carbons, which may be substituted or unsubstituted.

As used herein, the term “aryl” refers to an aromatic cyclic hydrocarbyl group having 5-14 ring carbon atoms, which may be mono- or polycyclic, which may contain fused rings, preferably 1 to 3 fused or unfused rings, and which may contain one or more heteroatoms, and/or which may be substituted or unsubstituted, as herein further described. Examples of “aryl” include, but are not limited to, phenyl, naphtyl, anthracyl, phenantryl, pyrrolyl, imidazolyl, thiophenyl, furanyl, oxazolyl, thiazolyl, pyridinyl, pyrimidinyl, pyrazinyl, triazinyl, and benzofuranyl, each of which may be substitute or unsubstituted. Typically, the term “aryl” refers to a substituted or unsubstituted phenyl.

As used herein, the term “acyl” refers to a group derived by the removal of one or more hydroxyl group from an oxoacid, preferably from a carboxylic acid. The acyl group according to the present invention is typically a saturated C₁₃₀ acyl, which may be substitute or unsubstituted.

As used herein the term “alkylene” refers to a bivalent saturated or unsaturated aliphatic radical deriving from a substituted or unsubstituted alkane by removal of two hydrogen atoms from different carbon atoms, preferably from the terminal carbon atoms. The alkylene according to the present invention, typically is a C₆₋₂₈ alkylene which may be saturated or contain one or more double and/or triple bonds, and/or which may be substituted or unsubstituted, as herein further described.

As used herein, the term “substituted” means that the group in question is substituted with a group which typically modifies the general chemical characteristics of the group in question. The substituents can be used to modify characteristics of the molecule, such as molecule stability, molecule solubility and the ability of the molecule to form crystals. The person skilled in the art will be aware of other suitable substituents of a similar size and charge characteristics, which could be used as alternatives in a given situation.

In connection with the terms “alkyl”, “aryl”, “acyl”, and “alkylene” the term substituted means that the group in question is substituted one or several times, preferably 1 to 3 times, with group(s) selected from hydroxy (which when bound to an unsaturated carbon atom may be present in the tautomeric keto form), oxo, C₁₋₆-alkoxy (i.e. C₁₋₆-alkyl-oxy), C₂₋₆-alkenyloxy, carboxy, oxo, C₁₋₆-alkoxycarbonyl, C₁₋₆-alkylcarbonyl, formyl, aryl, aryloxycarbonyl, aryloxy, arylamino, arylcarbonyl, heteroaryl, heteroarylamino, heteroaryloxycarbonyl, heteroaryloxy, heteroarylcarbonyl, amino, mono- and di(C₁₋₆alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, halogen, where any alkyl, alkoxy, and the like representing substituents may be substituted with hydroxy, C₁₋₆-alkoxy, C₂₋₆-alkenyloxy, carboxy, C₁₋₆-alkylcarbonylamino, halogen, C₁₋₆-alkylthio, C₁₋₆-alkyl-sulphonyl-amino, or guanidino.

The term “glycosyl moiety” when used herein is defined to encompass a moiety derived from a monosaccharide or from an oligosaccharide (more than one monosaccharide units), wherein the anomeric carbon of the monosaccharide or the anomeric carbon at the reducing end of the oligosaccharide is engaged in a glycosidic bond with another chemical entity, and the bond, if not further specified, may be an alpha or a beta glycosidic bond. A glycosyl moiety having more than one monosaccharide unit may represent a linear or a branched structure.

The monosaccharide unit can be any 5-9 carbon atom sugar, comprising aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglucosamine, N-acetylmannosamine, N-acetylgalactosamine, etc.), uronic acids, ketoaldonic acids (e.g. sialic acid). The monosaccharide unit can form different cyclic structures such as pyranose (six-membered) cyclic structures or furanose (five-membered) cyclic structures.

The glycosyl moieties according to the present invention may be illustrated in the following style: Galβ1-4Glc1-, wherein the dash (-) represents the point of attachment of the glycosyl moiety and wherein the glycosyl moiety may be linked via an alpha or a beta glycosidic bond, preferably a beta glycosidic bond.

The term, “oligosaccharide portion of a ganglioside” as used herein is defined to encompass glycosyl moieties deriving from gangliosides, wherein the anomeric carbon at the reducing end of the oligosaccharide portion of the ganglioside is engaged in a glycosidic bond with another chemical entity, the glycosidic bond may be an alpha or a beta glycosidic bond, preferably a beta glycosidic bond. In the context of the present invention the terms oligosaccharide portion and glycosyl moiety may be used interchangeably.

In the context of the present invention, the terms “about”, “around”, or “approximate” are applied interchangeably to a particular value (e.g. “a temperature of about 25° C.”, “a temperature of around 25° C.”, or “a temperature of approximate 25° C.”), or to a range (e.g. “an amount from about 1% to about 99%”, “an amount from around 1% to around 99%”, or “an amount from approximate 1% to approximate 99%”), to indicate a deviation from 0.1% to 10% of that particular value.

The term, “triazine-based acylating agent” as used herein refers to an activated derivate of a carboxylic acid, wherein the acidic hydroxyl group of the carboxylic acid is converted into a good leaving group via the replacement of the hydrogen atom with a 1,3,5-triazynyl group. Suitable triazine-based acylating agents for use in the context of the present invention are for example those represented by formulas (3)-(7) and (10)-(13), wherein the hydrogen atom of the acidic hydroxyl group of a carboxylic acid is replaced by a 4,6-dialkoxy-1,3,5-triazin-2-yl group, or by a 4,6-diaryloxy-1,3,5-triazin-2-yl group.

Accordingly, suitable triazine-based acylating agents for use in the context of the present invention carry one acyl group.

Triazine-based acylating agents carrying one acyl group can form several isomeric structures resulting from the migration of substituents on different positions of the 1,3,5-triazine ring. Depending on conditions such as temperature, solvent, and/or the use of a certain reagent such as a certain base, these isomeric structures can be isolated in a pure form or obtained as an isomeric mixture.

Accordingly, in the context of the present invention, the term “combination of triazine-based acylating agents” refers to a mixture comprising triazine isomeric structures which differ only in the position of the substituents on the triazine ring and can all serve as acylating agents.

In some embodiments, the triazine-based acylating agent of formula (3) is a triazine-based acylating agent is of formula (4).

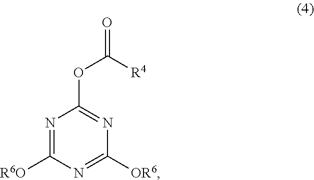

wherein
R⁴ is selected from hydrogen, a substituted or unsubstituted aryl, a heteroalkyl, a substituted or unsubstituted C₁₋₃₁ alkyl, preferably a substituted or unsubstituted C₉₋₃₁ alkyl,
R⁶ is selected from methyl, ethyl, 2,2,2-trifluoroethyl, and substituted or unsubstituted benzyl.

In some embodiments, the triazine-based acylating agent of formula (3) is a triazine-based acylating agent of formula (5).

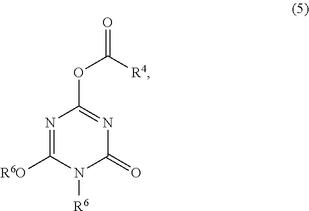

wherein
R⁴ is selected from hydrogen, a substituted or unsubstituted aryl, a heteroalkyl, a substituted or unsubstituted C₁₋₃₁ alkyl, preferably a substituted or unsubstituted C₉₋₃₁ alkyl,
R⁶ is selected from methyl, ethyl, 2,2,2-trifluoroethyl, and substituted or unsubstituted benzyl.

In some embodiments, the triazine-based acylating agent of formula (3) is a triazine based acylating agent of formulas (6), or (7):

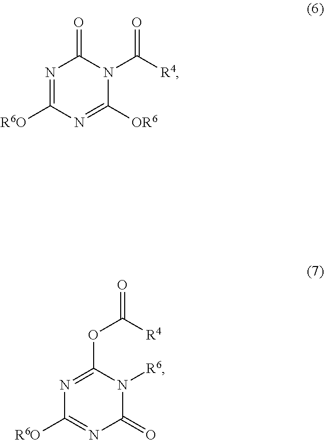

wherein,
R⁴ is selected from hydrogen, a substituted or unsubstituted aryl, a heteroalkyl, a substituted or unsubstituted C₁₋₃₁ alkyl, preferably a substituted or unsubstituted C₉₋₃₁ alkyl,
R⁶ is selected from methyl, ethyl, 2,2,2-trifluoroethyl, and substituted or unsubstituted benzyl.

In some embodiments, the triazine-based acylating agent is a triazine-based acylating of formula (18) or (19):

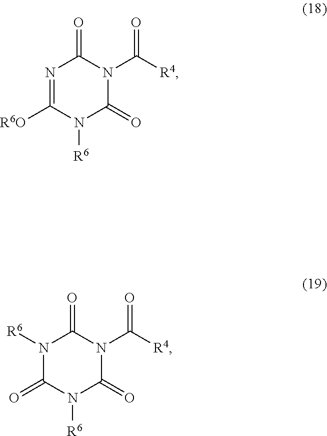

wherein
R⁴ is selected from hydrogen, a substituted or unsubstituted aryl, a heteroalkyl, a substituted or unsubstituted C₁₋₃₁ alkyl, preferably a substituted or unsubstituted C₉₋₃₁ alkyl, and
R⁶ is selected from methyl, ethyl, 2,2,2-trifluoroethyl, and substituted or unsubstituted benzyl.

In some embodiments, the triazine-based acylating agent of formula (3) is a combination comprising triazine-based acylating agents of formulas (4), (5), (6) and (7), and wherein each of said triazine-based acylating agent is present, in said combination, in the amount from about 1% to about 99%.

In some embodiments, the combination of triazine-based acylating agents of formulas (4), (5), (6) and (7), further comprises triazine based acylating agents of formulas (18), and (19), and wherein each of said triazine-based acylating agent is present, in said combination, in the amount from about 1% to about 99%.

Triazine-based acylating agents for use in the context of the present invention carry one acyl group preferably deriving from a carboxylic acid, and wherein the carboxylic acid is selected from formic acid, benzoic acid, acetic acid, or a fatty acid.

The acyl group carried by the triazine-based acylating for use in the context of the present invention may be illustrated in the following stile: —C(═O)R⁴, or may be represented by the acyl group of formula (20):

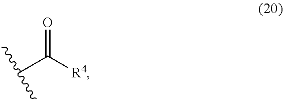

wherein
R⁴ is selected from hydrogen, a substituted or unsubstituted aryl, a heteroalkyl, a substituted or unsubstituted C₁₋₃₁ alkyl, preferably a substituted or unsubstituted C₉₋₃₁ alkyl,

In some preferred embodiments, R⁴ of the acyl group —C(═O)R⁴, or R⁴ of the acyl group of formula (20) is a substituted or unsubstituted C₉₋₃₁ alkyl selected from the group consisting of substituted or unsubstituted C₉₋₃₁ alkyl of formula (8) and (9):

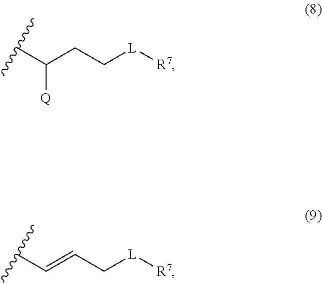

wherein
Q is selected from —H, —OH, —Cl, —Br, or —I,
L is a straight-chain C₆₋₂₈ alkylene, which may be saturated or contain one or more double bonds and/or triple bonds,
R⁷ is hydrogen, or —OR⁸, wherein R⁸ is hydrogen or a straight-chain C₂₋₃₀ acyl which may be saturated or contain one or more double bonds.

Accordingly in some preferred embodiments, the acyl group carried by the triazine-based acylating agents for use in the context of the present invention may be represented by an acyl group of formula (21) or (22):

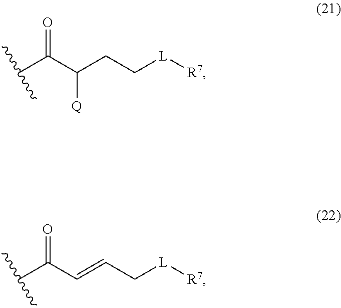

wherein
Q is selected from —H, —OH, —Cl, —Br, or —I,
L is a straight-chain C₆₋₂₈ alkylene, which may be saturated or contain one or more double bonds and
or triple bonds, R⁷ is hydrogen, or —OR⁸, wherein R⁸ is hydrogen or a straight-chain C₂₋₃₀ acyl which may be saturated or contain one or more double bonds.

In some embodiments, the substituted or unsubstituted C₉₋₃₁ alkyl is a substituted or unsubstituted C₉₋₃₁ alkyl of formula (8), wherein Q is —H, R⁷ is hydrogen, and L is a straight-chain saturated C₁₂₋₂₈ alkylene. Accordingly, in some embodiments, the acyl group carried by the triazine-based acylating agent for use in the context of the present invention is an acyl group of formula (21), wherein the acyl group of formula (21) is an acyl group deriving from a non-hydroxy fatty acids (N).

In some preferred embodiments, the acyl group carried by the triazine-based acylating agent for use in the context of the present invention, is an acyl group deriving from stearic acid [N (18:0)].

In some embodiments, the substituted or unsubstituted C₉₋₃₁ alkyl is a substituted or unsubstituted C₉₋₃₁ alkyl of formula (8), wherein Q is —OH, R⁷ is hydrogen, and L is a straight-chain saturated C₁₂₋₂₈ alkylene. Accordingly, in some embodiments, the acyl group carried by the triazine-based acylating agent for use in the context of the present invention is an acyl group of formula (21), wherein the acyl group of formula (21) is an acyl group deriving from an α-hydroxy fatty acids (A).

In some embodiments, the acyl group carried by the triazine-based acylating agent for use in the context of the present invention is an acyl group deriving from α-hydroxystearic acid [A(18:0)].

In some embodiments, the substituted or unsubstituted C₉₋₃₁ alkyl is a substituted or unsubstituted C₉₋₃₁ alkyl of formula (8), wherein Q is —H, L is a straight-chain saturated C₁₂₋₂₈ alkylene, and R⁷ is —OR⁸, wherein R⁸ is a linoleoyl group. Accordingly, in some embodiments, the acyl group carried by the triazine-based acylating agent for use in the context of the present invention is an acyl group of formula (21), wherein the acyl group of formula (21) is an acyl group deriving from a w-linoleoyloxy-fatty acid [E(18:2)O(12-28)].

In some embodiments, the acyl group carried by the triazine-based acylating agent for use in the context of the present invention is an acyl group of formula (21), wherein the acyl group pf formula (21) is selected from the group consisting of acyl groups of formula (23)-(31):

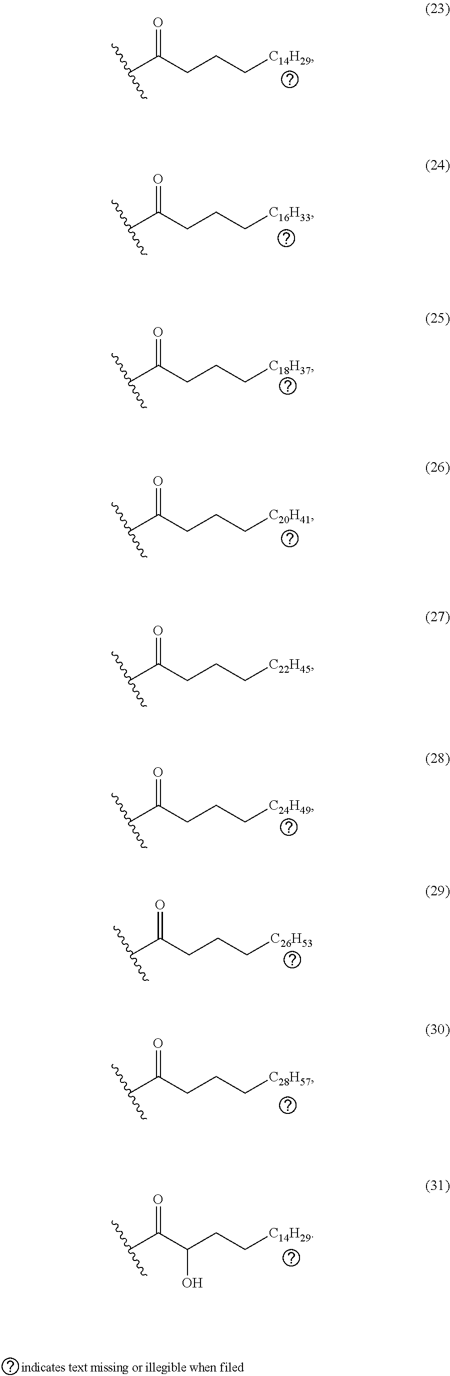

In some preferred embodiments, the triazine-based acylating agent of formula (3), is a triazin-based acylating agent of formulas (4), (5), (6) or (7), and wherein the triazine based acylating agents of formulas (4), (5), (6) and (7), are triazine based acylating agents of formulas (10), (11), (12), and (13), respectively:

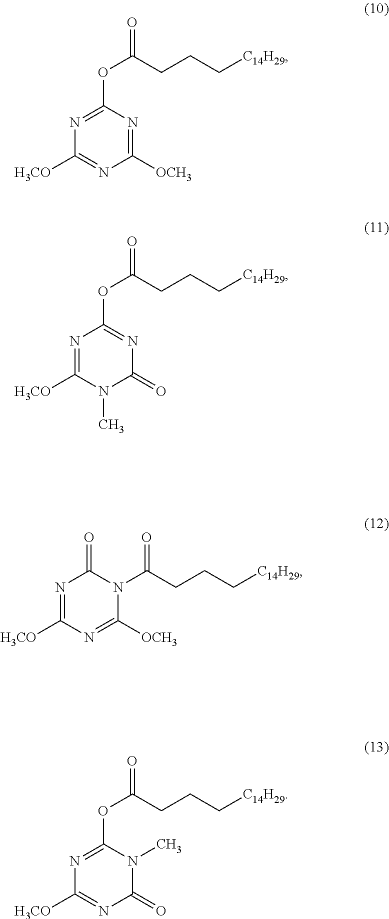

In some embodiments, the triazine-based acylating agent of formula (3) is a combination comprising triazine-based acylating agents of formulas (10), (11), (12) and (13), and wherein each of said triazines is present, in said combination, in the amount from about 1% to about 99%.

In some embodiments, the triazine-based acylating agent of formula (3) is a combination comprising triazine-based acylating agents of formulas (10) and (12), and wherein each of said triazines is present, in said combination, in the amount from about 1% to about 99%.

In some embodiments, the triazine-based acylating agent of formula (3) is a combination comprising triazine-based acylating agents of formulas (10) and (12), and wherein the triazine-based acylating agents of formulas (10) is present, in said combination, in the amount from about 5% to about 75%.

In some embodiments, the triazine-based acylating agent of formula (3) is a combination comprising triazine-based acylating agents of formulas (10) and (12), and wherein the triazine-based acylating agents of formulas (12) is present, in said combination, in the amount from about 5% to about 75%.

In some embodiments, the triazine-based acylating agent of formula (3) is a combination comprising triazine-based acylating agents of formulas (11) and (13), and wherein each of said triazines is present, in said combination, in the amount from about 1% to about 99%.

In some preferred embodiments, the triazine-based acylating agent of formula (3) is a combination comprising triazine-based acylating agents of formulas (11) and (13), and wherein the triazine-based acylating agents of formulas (11) is present, in said combination, in the amount from about 5% to about 50%.

In some preferred embodiments, the triazine-based acylating agent of formula (3) is a combination comprising triazine-based acylating agents of formulas (11) and (13), and wherein the triazine-based acylating agents of formulas (13) is present, in said combination, in the amount from about 5% to about 50%.

For those embodiments, describing a combination of triazine-based acylating agents, the amount of the triazine-based acylating agents comprised in the combination is typically determined via ¹H NMR spectroscopy.

In a typical procedure:
i. characteristic signals deriving from each of the triazine-based acylating agents of the combination are identified,
ii. the signals are integrated,
iii. the ratio of the integrals is calculated and utilized to the define the amount of the different triazine-based acylating agents of the combination.

The person skilled in the art will understand that the amount of the triazine-based acylating agents of the combination may represent a molar ratio or mol %, or a weight ratio or wt %. The person skilled in the art will also understand that the molar ratio or mol %, or the weight ratio or wt % of the triazine-based acylating agents of the combination, may vary over time due interconversion between the different isomeric structures of the triazine-based acylating agent.

In some preferred embodiments, R⁶ of the triazine-based acylating agents of formulas (3)-(7), (10)-(13), (18), or (19) is methyl.

Triazine-based acylating agents according to the present invention, or their combination thereof, may be produced by standard methods known to the skilled person. A method for the synthesis of triazine-based acylating agent is e.g. described by Z. J. Kamiński, J. prakt. Chem. 1990, 4, 579-583.

Alternatively, triazine-based acylating agents or combinations thereof may be synthesized according to the method of the present invention.

The triazine-based acylating agents according to the present invention may be utilized or produced in different polymorphic forms. Polymorphic forms as referred to herein can include crystalline and amorphous forms as well as solvate and hydrate forms, which can be further characterized as follows:
i. Crystalline forms have different arrangements and/or conformations of the molecules in the crystal lattice.
ii. Amorphous forms consist of disordered arrangements of molecules that do not possess a distinguishable crystal lattice.
iii. Solvates are crystal forms containing either stoichiometric or non-stoichiometric amounts of a solvent. If the incorporated solvent is water, the solvate is commonly known as a hydrate.

In some embodiments, the triazine-based acylating agents are present as solvates.

In some embodiments, the triazine-based acylating agents are present as hydrates, such as in the form of monohydrates, dihydrates or trihydrates.

In some embodiments, the triazine-based acylating agents are present in a crystalline form.

In some embodiments, the triazine-based acylating agents are present in an amorphous form.

The present invention describes a method for the production of a sphingolipid of formula (1), wherein the triazine-based acylating agent according to the present invention or a combination of triazine-based acylating agents according to the present invention, is reacted with a lysosphingolipid of formula (2).

In some preferred embodiments, a triazine-based acylating agent of formula (10), is reacted with a lysosphingolipid of formula (2).

In some preferred embodiments, a triazine-based acylating agent of formula (11), is reacted with a lysosphingolipid of formula (2).

In some embodiments, a combination comprising triazine-based acylating agents of formulas (10), (11), (12) and (13), is reacted with a lysosphingolipid of formula (2), and wherein each of said triazine-based acylating agent is present, in said combination, in the amount from about 1% to about 99%.

In some embodiments, a combination comprising triazine-based acylating agents of formulas (10) and (12), is reacted with a lysosphingolipid of formula (2), and wherein each of said triazine-based acylating agent is present, in said combination, in the amount from about 1% to about 99%.

In some embodiments, a combination comprising triazine-based acylating agents of formulas (10) and (12), is reacted with a lysosphingolipid of formula (2), and wherein the triazine-based acylating agents of formulas (10) is present, in said combination, in the amount from about 5% to about 75%.

In some embodiments, a combination comprising triazine-based acylating agents of formulas (10) and (12), is reacted with a lysosphingolipid of formula (2), and wherein the triazine-based acylating agents of formulas (12) is present, in said combination, in the percentage from about 5% to about 75%.

In some embodiments, a combination comprising triazine-based acylating agents of formulas (11) and (13), is reacted with a lysosphingolipid of formula (2), and wherein each of said triazine-based acylating agent is present, in said combination, in the amount from about 1% to about 99%.

In some preferred embodiments, a combination comprising triazine-based acylating agents of formulas (11) and (13), is reacted with a lysosphingolipid of formula (2), and wherein the triazine-based acylating agents of formulas (11) is present, in said combination, in the amount from about 5% to about 50%.

In some embodiments, a combination comprising triazine-based acylating agents of formulas (11) and (13), is reacted with the lysosphingolipid of formula (2) and wherein the triazine-based acylating agents of formulas (13) is present, in said combination, in the amount from about 5% to about 50%.

The term “lysosphingolipid” when used herein refers to a sphingolipid breakdown product which lack the amide-linked fatty acyl group at the 2-position of the sphingoid base backbone. Suitable lysosphingolipids, for use in the context of the present invention, are sphingoid bases or glycosylated sphingoid bases and are represented by a lysosphingolipid of formula (2).

Lysosphingolipids for use in the context of the present invention are preferably obtained via synthetic and/or biotechnological approaches such as those described in WO 2021170624 A2, or in WO2019238970 A1, WO2022158993 A1, or by Sarmientos et al., Eur. J. Biochem. 1986, 160,527-535.

In some embodiments, the lysosphingolipid of formula (2) is a lysosphingolipid of formula (14), or a salt thereof:

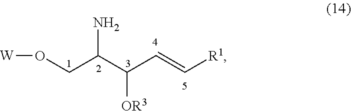

wherein
W is hydrogen or a glycosyl moiety,
R¹ is H, aryl, or a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of a hydroxyl group, an alkoxy group, an acyloxy group, an acylamido group, a thiol, a thioether or a phosphorus-containing functional group, and
R³ is H, a substituted or unsubstituted C₁₋₆ alkyl, or a substituted or unsubstituted C₁₋₆ acyl.

In some embodiments, the stereochemical configuration of the C-2, C-3, and C-4 carbon atoms of the lysosphingolipid of formula (14) is (2S,3R,4E).

In some preferred embodiments, the lysosphingolipid of formula (14) is D-erythro-sphingosine.

In some embodiments, the lysosphingolipid of formula (14) is 6-hydroxy-D-erythro-sphingosine.

In some embodiments, the lysosphingolipid of formula (2) is a lysosphingolipid of formula (15), or a salt thereof:

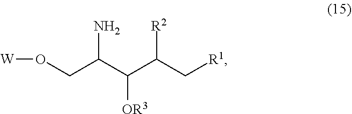

wherein
W is hydrogen or a glycosyl moiety,
R¹ is H, aryl, or a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of a hydroxyl group, an alkoxy group, an acyloxy group, an acylamido group, a thiol, a thioether or a phosphorus-containing functional group,
R² is H or —OR⁵, wherein R⁶ is selected from hydrogen, a substituted or unsubstituted C₁₋₆ alkyl, or a substituted or unsubstituted C₁₋₆ acyl,
R³ is H, a substituted or unsubstituted C₁₋₆ alkyl, or a substituted or unsubstituted C₁₋₆ acyl.

In some embodiments the stereochemical configuration of the C-2, C-3, and C-4 carbon atoms of the lysosphingolipid of formula (15) is (2S,3S,4R).

In some preferred embodiments, the lysosphingolipid of formula (15) is D-ribo-phytosphingosine.

In some preferred embodiments, the lysosphingolipid of formula (15) is DL-erythro-dihydrosphingosine.

In some embodiments, lysosphingolipids of formula (2), (14), and (15) may be produced or utilized in the form of salts, preferably in the form of pharmaceutical acceptable salts.

In some embodiments, the salts of compounds of formula (2), (14), and (15), may be formed from the following acids: hydrochloric acid, hydrobromic acid, sulfuric acid, phosphoric acid, polyphosphoric acid, acetic acid, camphor sulfonic acid, p-toluene sulfonic acid, methane sulfonic acid, trifluoromethanesulfonic acid, perchloric acid.

Typically, the lysosphingolipid, and the triazine-based acylating agent, or a composition thereof are reacted in a polar solvent such as methanol, ethanol, propanol, isopropanol, butanol, or isobutanol.

In some preferred embodiment, the reaction is performed in methanol. In some embodiment the reaction is performed in a mixture of one or more polar solvents, such as a mixture of methanol and ethanol, methanol and propanol, methanol and isopropanol, methanol and butanol, methanol and isobutanol, or methanol and water.

In some embodiments, the lysosphingolipid and the triazine-based acylating agent, or the composition thereof are reacted in the presence of a base such as NaOH, KOH, LiOH, Ca(OH)₂, triethylamine, N,N-diisopropylethylamine, and pyridine. In some preferred embodiments, the based is selected from NaOH, or KOH.

The purity of the triazine-based acylating agent according to the present invention, or the combination thereof may vary from a purity of about 99% to a purity of about 70%.

The reaction between the lysosphingolipid and the triazine-based acylating agent, or the composition thereof is typically performed at temperature from about 25° C. to about 65° C. Accordingly, in some embodiments, the reaction is performed at a temperature of about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C. Preferably, the reaction between the lysosphingolipid and the triazine-based acylating agent, or a composition thereof is performed at temperature from about 45° C. to about 55° C. Accordingly, in some preferred embodiments the reaction is performed at a temperature of 45° C., 46° C., 47° C., 48° C., 49° C., 50° C., 51° C., 52° C., 53° C., 54° C., or 55° C.

The components of the reactions of the invention may be combined in any order, and it will be appreciated that the order of combining the reactants may be adjusted as needed. For example, the lysosphingolipid may be added to a solution of the triazine-based acylating agent, or the composition thereof. As another example the triazine-based acylating agent, or the composition thereof may be added to a solution of the lysosphingolipid. As yet another example, a solvent may be added to a flask containing the lysosphingolipid and the triazine-based acylating agent, or the composition thereof.

The lysosphingolipid and the triazine-based acylating agent, or the composition thereof, as well as any other reagent used during the reaction may be added to the reaction either as a solid or dissolved in a solvent, and in any quantities and manner effective for the intended result of the reaction.

In some embodiments, the present invention discloses a method for the production of a sphingolipid of formula (1), wherein the method further comprising steps of producing a triazine-based acylating agents of formula (3), or a combination thereof, and wherein the triazine-based acylating agents of formula (3), or the combination thereof is produced via the steps of:
reacting a carboxylic acid of formula (16):

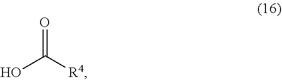

wherein
R⁴ is selected from hydrogen, a substituted or unsubstituted aryl, a heteroalkyl, a substituted or unsubstituted C₁₋₃₁ alkyl, preferably a substituted or unsubstituted C₉₋₃₁ alkyl, with a compound of formula (17):

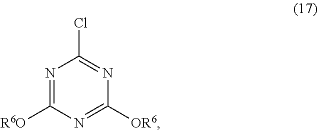

wherein R⁶ is as is selected from methyl, ethyl, 2,2,2-trifluoroethyl, and substituted or unsubstituted benzyl,
in the presence of an organic base, thereby producing the triazine-based acylating agents of formula (3), or the combination thereof;
isolating the triazine-based acylating agent of formula (3) via precipitation;
and wherein the steps of producing the triazine-based acylating agent, or the combination thereof are performed in a non-halogenated solvent.

In some embodiments, the present invention discloses a method for the production of a sphingolipid of formula (1), wherein the method comprising steps of:
reacting a carboxylic acid of formula (16):

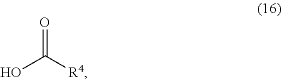

wherein
R⁴ is selected from hydrogen, a substituted or unsubstituted aryl, a heteroalkyl, a substituted or unsubstituted C₁₋₃₁ alkyl, preferably a substituted or unsubstituted C₉₋₃₁ alkyl,
with a compound of formula (17):

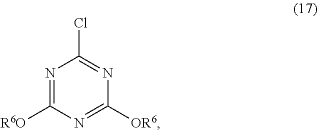

wherein R⁶ is as is selected from methyl, ethyl, 2,2,2-trifluoroethyl, and substituted or unsubstituted benzyl,
in the presence of an organic base, thereby producing a triazine-based acylating agents of formula (3), or a combination thereof:

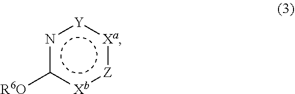

wherein

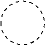

represents a conjugated system of bonds such that either two or three double bonds are present in the ring;
Y is selected from C(O—C(═O)R⁴), or C(═O),
X^{a} is selected from N, NR⁶, or N(C(═O)R⁴)
X^{b} is selected from N, or NR⁶
Z is selected from C(═O), or C(OR⁶),
and provided that:
when Y is C(O—C(═O)R⁴), Z is C(OR⁶), X^{a} and X^{b} are N, and three double bonds are present in the ring, or
when Y is C(O—C(═O)R⁴), Z is C(═O), one of X^{a} and X^{b} is N and the other group is NR⁶, and two double bonds are present in the ring, or
when Y is C(═O), Z is C(OR⁶), X^{a} is N(C(═O)R⁴), X^{b} is N, and two double bonds are present in the ring,
and wherein
R⁴ is selected from hydrogen, a substituted or unsubstituted aryl, a heteroalkyl, a substituted or unsubstituted C₁₋₃₁ alkyl, preferably a substituted or unsubstituted C₉₋₃₁ alkyl,
R⁶ is selected from methyl, ethyl, 2,2,2-trifluoroethyl, and substituted or unsubstituted benzyl;
isolating the triazine-based acylating agent of formula (3), or the combination thereof via precipitation;
and wherein the steps of producing the triazine-based acylating agent of formula (3), or the combination thereof are performed in a non-halogenated solvent.
reacting a lysosphingolipid of formula (2):

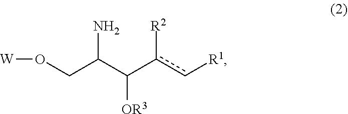

wherein W, the bond [symbol] , R¹, R² and R³ are as defined as for the sphingolipid of formula (1), with the triazine-based-acylating agent of formula (3), or the combination thereof, thereby producing the sphingolipid of formula (1).

Typically, the step of reacting the carboxylic acid of formula (16) with the compound of formula (17) is performed in the presence of an organic base. In some embodiments, the organic base is selected from 4-methylmorpholine, 1,4-diazabicyclo[2.2.2]octane, preferably 4-methylmorpholine.

Typically, the steps of the method for the synthesis of the triazine-based acylating agents of formula (3), or the combination thereof are performed in the same non-halogenated solvent. The non-halogenated solvent is preferably selected from a ketone, an alcohol, or an aliphatic hydrocarbon. In some embodiments, the non-halogenated solvent is a ketone selected from acetone, diethyl ketone, methyl isobutyl ketone, or butan-2-one, preferably acetone. In some embodiments, when the solvent is a ketone, water may be added to the reaction mixture. In some embodiments, the non-halogenated solvent is an alcohol selected from methanol, ethanol, propanol, isopropanol, butanol, isobutanol, preferably methanol, or a mixture thereof. In some embodiments, the non-halogenated solvent is an aliphatic hydrocarbon selected from petroleum ether, hexane, or an isomeric mixture thereof, n-heptane, or an isomeric mixture thereof, octane, or an isomeric mixture thereof. In some embodiments, the non-halogenated solvent is a mixture of two alcohols, and wherein the mixture of two alcohols is preferably selected from a mixture of methanol and ethanol, methanol and propanol, methanol and isopropanol, methanol and butanol, or methanol and isobutanol, preferably a mixture of methanol and ethanol.

In some embodiments, the step of reacting the carboxylic acid of formula (16) with the compound of formula (17), is performed at a temperature between about 30° C. and about 100° C., preferably between about 30° C. and about 55° C.

In some embodiments, step isolating the triazine-based acylating agents of formula (3), or the combination thereof is performed at a temperature between about −20° C. and about 25° C., preferably at a temperature between about −10° C. and about 25° C., even more preferably at a temperature between about 5° C. and about 25° C.

The purity of the triazine-based acylating agent according to the present invention, or the combination thereof may vary from a purity of about 99% to a purity of about 70%.

In some embodiments, stearic anhydride (from about 8 mol % to about 30 mol %) may be present in combination with the triazine-based acylating agent according to the present invention.

The reaction between the triazine-based acylating agents according to the present invention and the lysosphingolipid of formula (2), (14), or (15), or the reaction between a composition of triazine-based acylating agents according to the present invention, and the lysosphingolipid of formula (2), (14), or (15), results in the selective N-acylation of the amino group at the C-2 carbon atom of the lysosphingolipid of (2), (14), or (15), thereby producing a sphingolipid of formula (1).

In some embodiments, the sphingolipid of formula (1) is a sphingolipid of formula (32), or (33):

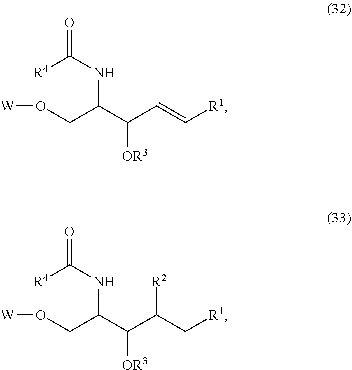

wherein
W is hydrogen or a glycosyl moiety,
R¹ is H, aryl, or a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of a hydroxyl group, an alkoxy group, an acyloxy group, an acylamido group, a thiol, a thioether or a phosphorus-containing functional group,
R² is H or —OR⁵, wherein R⁶ is selected from hydrogen, a substituted or unsubstituted C₁₋₆ alkyl, or a substituted or unsubstituted C₁₋₆ acyl,
R³ is H, a substituted or unsubstituted C₁₋₆ alkyl, or a substituted or unsubstituted C₁₋₆ acyl,
R⁴ is selected from hydrogen, a substituted or unsubstituted aryl, a heteroalkyl, a substituted or unsubstituted C₁₋₃₁ alkyl, preferably a substituted or unsubstituted C₉₋₃₁ alkyl.

Sphingolipids of formulas (1), (32), or (33) carry an acyl group, preferably deriving from a carboxylic acid, and wherein the carboxylic acid is selected from formic acid, benzoic acid, acetic acid, or a fatty acid.

The acyl group carried by the sphingolipids of formulas (1), (32), or (33) may be represented by the acyl group of formula (20):

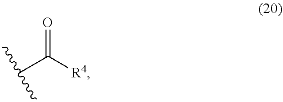

wherein
R⁴ is selected from hydrogen, a substituted or unsubstituted aryl, a heteroalkyl, a substituted or unsubstituted C₁₋₃₁ alkyl, preferably a substituted or unsubstituted C₉₋₃₁ alkyl,

In some preferred embodiments, R⁴ of the acyl group of formula (20) is a substituted or unsubstituted C₉₋₃₁ alkyl selected from the group consisting of substituted or unsubstituted C₉₋₃₁ alkyl of formula (8) and (9):

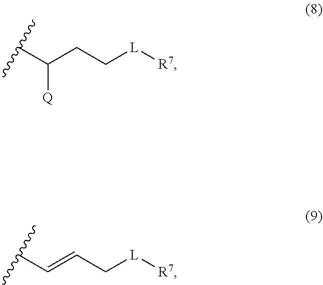

wherein
Q is selected from —H, —OH, —Cl, —Br, or —I,
L is a straight-chain C₆₋₂₈ alkylene, which may be saturated or contain one or more double bonds and or triple bonds,
R⁷ is hydrogen, or —OR⁸, wherein R⁸ is hydrogen or a straight-chain C₂₋₃₀ acyl which may be saturated or contain one or more double bonds.

Accordingly in some preferred embodiments, the acyl group carried by the sphingolipids of formulas (1), (32), or (33) may be represented by an acyl group of formula (21) or (22):

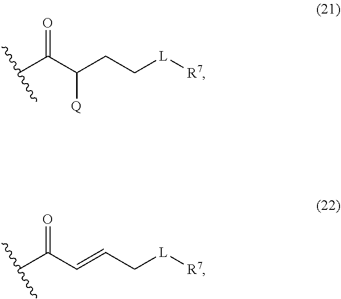

wherein
Q is selected from —H, —OH, —Cl, —Br, or —I,
L is a straight-chain C₆₋₂₈ alkylene, which may be saturated or contain one or more double bonds and or triple bonds,
R⁷ is hydrogen, or —OR⁸, wherein R⁸ is hydrogen or a straight-chain C₂₋₃₀ acyl which may be saturated or contain one or more double bonds.

In some embodiments, the substituted or unsubstituted C₉₋₃₁ alkyl is a substituted or unsubstituted C₉₋₃₁ alkyl of formula (8), wherein Q is —H, R⁷ is hydrogen, and L is a straight-chain saturated C₁₂₋₂₈ alkylene. Accordingly, in some embodiments, the acyl group carried by the sphingolipids of formulas (1), (32), or (33) is an acyl group of formula (21), wherein the acyl group of formula (21) is an acyl group deriving from a non-hydroxy fatty acid (N).

In some preferred embodiments, the acyl group carried by the sphingolipids of formulas (1), (32), or (33), is an acyl group deriving from stearic acid [N (18:0)].

In some embodiments, the substituted or unsubstituted C₉₋₃₁ alkyl is a substituted or unsubstituted C₉₋₃₁ alkyl of formula (8), wherein Q is —OH, R⁷ is hydrogen, and L is a straight-chain saturated C₁₂₋₂₈ alkylene. Accordingly, in some embodiments, the acyl group carried by the sphingolipids of formulas (1), (32), or (33) is an acyl group of formula (21), wherein the acyl group of formula (21) is an acyl group deriving from an α-hydroxy fatty acids (A).

In some embodiments, the acyl group carried by the sphingolipids of formulas (1), (32), or (33) is an acyl group deriving from α-hydroxystearic acid [A(18:0)].

In some embodiments, the substituted or unsubstituted C₉₋₃₁ alkyl is a substituted or unsubstituted C₉₋₃₁ alkyl of formula (8), wherein Q is —H, L is a straight-chain saturated C₁₂₋₂₈ alkylene, and R⁷ is —OR⁸, wherein R⁸ is a linoleoyl group. Accordingly, in some embodiments, the acyl group carried by the sphingolipids of formulas (1), (32), or (33) is an acyl group of formula (21), wherein the acyl group of formula (21) is an acyl group deriving from a w-linoleoyloxy-fatty acid [E(18:2)O(12-28)].

In some embodiments, the acyl group carried by the sphingolipids of formulas (1), (32), or (33) is an acyl group of formula (21), wherein the acyl group pf formula (21) is selected from the group consisting of acyl groups of formula (23)-(31):

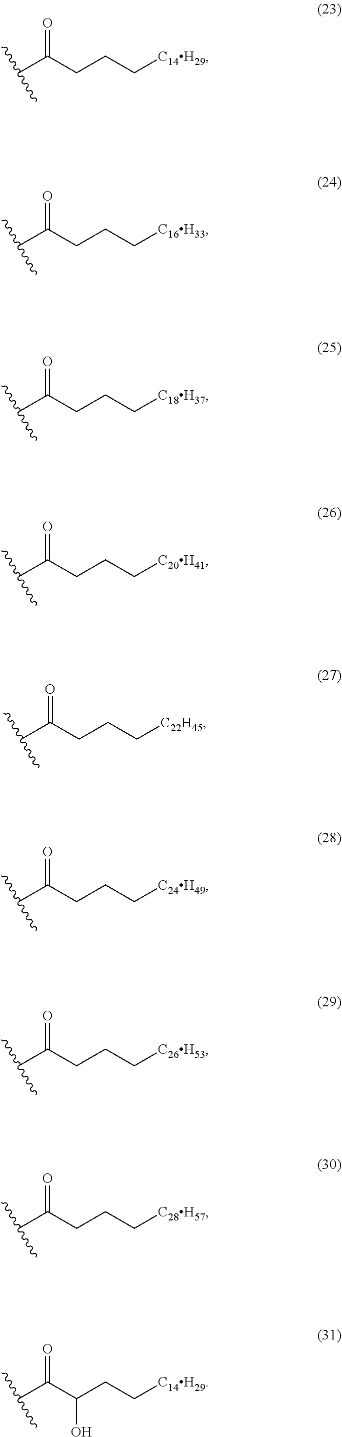

In some embodiments for the sphingolipid of formula (1), (32), or (33), W is hydrogen, R¹ is a substituted or unsubstituted C₁₃ alkyl, R² is hydrogen or —OH, and R³ is hydrogen. Accordingly in some embodiments, the sphingolipid of formula (1), (32), or (33) is a ceramide. Ceramides denote, in the context of the present invention, naturally occurring ceramides, analogues thereof or derivatives thereof. Preferred ceramides are those naturally occurring in humans. Naturally occurring human ceramides (CERs) include, but are not limited to, CER[NS], CER[AS], CER[EOS], CER[NH], CER[AH], or CER[EOH], CER[NP], CER[AP], or CER[EOP], CER[NdS], CER[AdS], or CER[EOdS], wherein letters in brackets refer to the shorthand nomenclature developed by Motta et al., Biochim Biophys Acta., 1993, 1182:147-151 and expanded by Rabionet et al., Biochim Biophys Acta, 2014, 1841:422-434, and by Masukawa et al., Journal of Lipid Research, 2008, 49, 1466-1476. Particularly, the letters N, A, and EO represent non-hydroxy fatty acids (N), alpha-hydroxy fatty acids (A), and omega-linoleoyloxy fatty acids (EO), respectively, wherein the number of fatty acid carbons and unsaturations may be expressed in parentheses following the letters of N, A, E, and O. The letters, S, H, P, and dS represent D-erythro-sphingosine (S), 6-hydroxy-D-erythro-sphingosine (H), D-ribo-phytosphingosine (P), DL-erythro-dihydrosphingosine (dS), respectively, wherein the number of sphingoid carbons may be expressed in parenthesis following the letters S, H, P, and dS. Ceramides, CER[NdS], CER[AdS], or CER[EOdS], may also be referred to as CER[NG], CER[AG], or CER[EOG], respectively, wherein the letter G represent the INCI name for DL-erythro-dihydrosphingosine.

In some embodiment, the sphingolipid of formula (1) is a sphingolipid of formula (32), wherein the sphingolipid of formula (32) is a ceramide selected from CER[N (16:0) S (18)], CER[A (16:0) S (18)], CER[N (18:0) S (18)], CER[A (18:0) S (18)], CER[N (20:0) S (18)], CER[A (20:0) S (18)],

In some embodiment, the sphingolipid of formula (1) is a sphingolipid of formula (32), wherein the sphingolipid of formula (32) is a ceramide selected from CER[N (16:0) H (18)], CER[A (16:0) H (18)], CER[N (18:0) H (18)], CER[A (18:0) H (18)], CER[N (20:0) H (18)], CER[A (20:0) H (18)].

In some embodiment, the sphingolipid of formula (1) is a sphingolipid of formula (33), wherein the sphingolipid of formula (33) is a ceramide selected from CER[N (16:0) P (18)], CER[A (16:0) P (18)], CER[N (18:0) P (18)], CER[A (18:0) P (18)], CER[N (20:0) P (18)], CER[A (20:0) P (18)].

In some embodiment, the sphingolipid of formula (1) is a sphingolipid of formula (33), wherein the sphingolipid of formula (33) is a ceramide selected from CER[N (16:0) dS (18)], CER[A (16:0) dS (18)], CER[N (18:0) dS (18)], CER[A (18:0) dS (18)], CER[N (20:0) dS (18)], CER[A (20:0) dS (18)].

In some preferred embodiments, W of the lysosphingolipid of formula (2), (14), or (15), and W of the sphingolipid of formula (1), (32), or (33) is a glycosyl moiety, and wherein the glycosyl moiety is selected from Glc1-, Gal1-, Galβ1-4Glc1-.

In some embodiments, W of the lysosphingolipid of formula (2), (14), or (15), and W of the sphingolipid of formula (1), (32), or (33) is a glycosyl moiety, and wherein the glycosyl moiety is the oligosaccharide portion of a ganglioside selected from GM1a, GM1b, GD1a, GD1b, GD3, GT1b, GT3, GQ1b, GM3, GM4, preferably GM4, GM3, or GD3, more preferably GM3.

In the context of the present invention the oligosaccharide portion of from GM1a, GM1b, GD1a, GD1b, GD3, GT1b, GT3, GQ1b, GM3, GM4 may be represented by the following formulas:

Galβ1-3GalNAcβ1-4(Neu5Acα2-3)Galβ1-4Glc1-,
Neu5Acα2-3Galβ1-3GalNAcβ1-4Galβ1-4Glc1-,
Neu5Acα2-3Galβ1-3GalNAcβ1-4(Neu5Acα2-3)Galβ1-4Glc1-,
Galβ1-3GalNAcβ1-4(Neu5Acα2-8Neu5Acα2-3)Galβ1-4Glc1-,
Neu5Acα2-8Neu5Acα2-3Galβ1-4Glc1-,
Neu5Acα2-3Galβ1-3GalNAcβ1-4(Neu5Acα2-8Neu5Acα2-3)Galβ1-4Glc1-,
Neu5Acα2-8Neu5Acα2-8Neu5Acα2-3Galβ1-4Glc1-,
Neu5Acα2-8Neu5Acα2-3Galβ1-3GalNAcβ1-4(Neu5Acα2-8Neu5Acα2-3)Galβ1-4Glc1-,
Neu5Acα2-3Galβ1-4Glc1-,
Neu5Acα2-3Gal1-,

respectively.

In some embodiments, W of the lysosphingolipid of formula (2), (14), or (15), and W of the sphingolipid of formula (1), (32), or (33) is a glycosyl moiety, wherein the glycosyl moiety is that of a human milk oligosaccharide, and wherein the human milk oligosaccharide is preferably selected from LNT, LNnT, LNH, LNnH, 2′FL, 3FL, DFL, LNFP-1, LNFP-II, LNFP-III, LNFP-V, LNDFH-1, 3SL, 6′SL, FSL, LSTa, LSTb, LSTc, and DSLNT.

In the context of the present invention the oligosaccharide portion of LNT, LNnT, LNH, LNnH, 2′FL, 3FL, DFL, LNFP-1, LNFP-II, LNFP-Ill, LNFP-V, LNDFH-1, 3SL, 6′SL, FSL, LSTa, LSTb, LSTc, and DSLNT may be represented by the following formulas:

Galβ1-3GlcNAcβ1-3Galβ1-4Glc1-,
Galβ1-4GlcNAcβ1-3Galβ1-4Glc1-,
Galβ1-3GlcNAcβ1-3(Galβ1-4GlcNAcβ1-6)Galβ1-4Glc1-,
Galβ1-4GlcNAcβ1-3Galβ1-4GlucNAcβ1-3Galβ1-4Glc1-,
Fuca1-2Galβ1-4Glc1-,
Galβ1-4(Fucα1-3)Glc1-,
Fuca1-2Galβ1-4(Fucα1-3)Glc1-,
Fuca1-2Galβ1-3GlcNAcβ1-3Galβ1-4Glc1-,
Galβ1-3(Fucα1-4)GlcNAcβ1-3Galβ1-4Glc1-,
Galβ1-3(Fucα1-3)GlcNAcβ1-3Galβ1-4Glc1-,
Galβ1-3GlcNAcβ1-3Galβ1-4(Fucα1-3)Glc1-,
Fuca1-2Galβ1-3(Fucα1-4)GlcNacβ1-3Galβ1-4Glc1-,
Neu5Acα2-3Galβ1-4Glc1-,
Neu5Acα2-6Galβ1-4Glc1-,
Neu5Acα2-3Galβ1-4(Fucα1,3)Glc1-,
Neu5Acα2-3Galβ1-3GlcNAcβ1-3Galβ1-4Glc1-,
Galβ1-3(Neu5Acα2-6)GlcNAcβ1-3Galβ1-4Glc1-,
Neu5Acα2-6Galβ1-3GlcNAcβ1-3Galβ1-4Glc1-,
Neu5Acα2-3Galβ1-3(Neu5Acα2-6)GlcNAcβ1-3Galβ1-4Glc1-,

respectively.

In some embodiments, W of the lysosphingolipid of formula (2), (14), or (15), and W of the sphingolipid of formula (1), (32), or (33) is a glycosyl moiety, and wherein the glycosyl moiety is the oligosaccharide portion of a glycosphingolipid selected from the gala series [SP0509], the neogala series, the globo series [SP0502], the isoglobo series [SP0506], the lacto series [SP0504], the neolacto series [SP0505], the arthro series [SP0508], the muco series, the schisto series, the spirometo series, or protected analogs thereof.

Wherein the bracketed alphanumeric string indicates a link contained on the website https://www.lipidmaps.org/, wherein detailed chemical formulas and structural information for the corresponding compounds can be found.

In the context of the present invention lysosphingolipid of formula (2), (14), or (15), wherein W is a glycosyl moiety may also be referred to as glycosylated sphingoid bases.

In the context of the present invention, sphingolipids of formula (1), (32), or (33) wherein W is a glycosyl moiety may also be referred to as glycosphingolipids or glycosylated ceramides.

In the context of the present invention, glycosyl moiety may be linked via an alpha or a beta glycosidic bond, preferably a beta glycosidic bond.

In some preferred embodiments, the sphingolipid of formula (1) is a sphingolipid of formula (32), and wherein the sphingolipid of formula (32) is selected from the group consisting of sphingolipids of formulas (34)-(38):

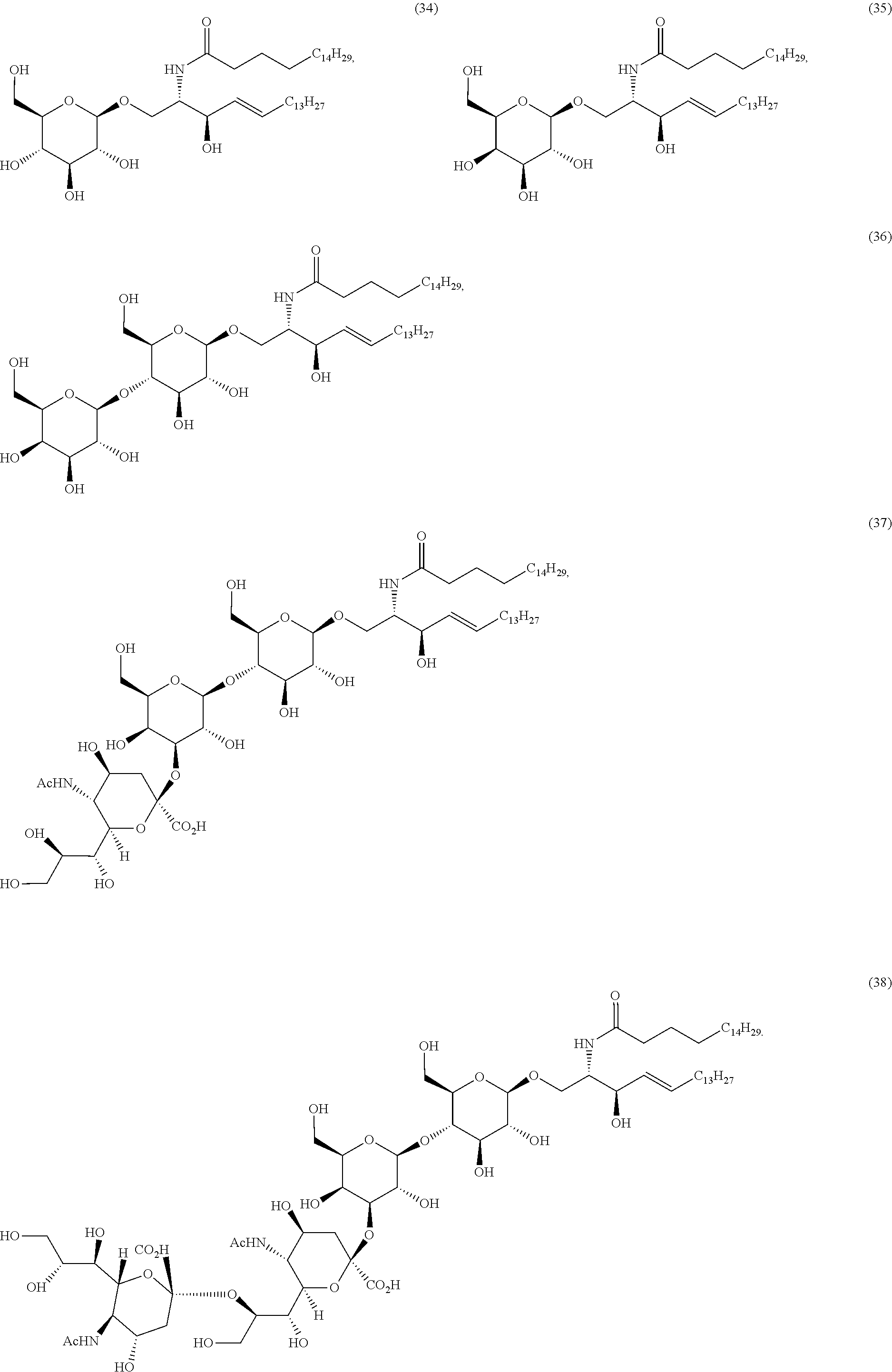

The skilled person will understand that in formulas showing a specific compound, unless the chemical formula expressly describes a carbon atom having a particular stereochemical configuration, the formula is intended to cover compounds where such a stereocenter has an R or an S configuration, or wherein a double bond has an E or a Z configuration.

### EXAMPLES

### General Methods and Material:

¹H NMR and ¹³C NMR was recorded with a Bruker WM-300S (300/75.1 MHz) spectrometer. ¹H and ¹³C chemical shifts are given in ppm (δ) relative to CDCl₃ (δ=7.26, or δ=77.2) as internal standard. LCMS analysis was performed with a Shimadzu ECO 2020 LC system coupled with a Shimadzu LCMS-2020 system. TLC-analysis was performed with silica gel TLC-plates (Merck, Silica gel, F254) with detection by carring (±140° C.) with ammonium molybdate (25 g/L) and cerium ammonium sulfate (10 g/L) in 10% H₂SO₄. Lysosphingolipids such as for example D-erythro-sphingosine, β-D-galactopyranosyl-(1→4)-β-D-glucopyranosyl-(1→1′)-D-erythro-sphingosine, or α-N-acetylneuraminosyl-(2→3)-O-β-D-galactopyranosyl-(1→4)-β-D-glucopyranosyl-(1→1′)-D-erythro-sphingosine were synthesized according to procedure described in WO 2021170624 A2, WO2019238970 A1, WO2022158993 (A1) or by Sarmientos et al., Eur. J. Biochem. 1986, 160,527-535. Fatty acids were purchased by established manufacturers.

### Example 1: General Procedure for the Synthesis of Triazine-Based Acylating Agents

A fatty acid (1 eq.) was suspended in n-heptane, acetone, or a mixture of ethanol/methanol. 2-Chloro-4,6-dimethoxy-1,3,5-triazine (1 eq.), and 4-methylmorpholine (1 eq.) were added to the suspension/solution. The reaction mixture was stirred at a temperature between 20° C. to 50° C. until a TLC-analysis showed complete consumption of the starting material.

The reaction mixture was cooled down to a temperature between 5° C. to 30° C. When acetone was used as solvent, water was added to the reaction mixture prior cooling. A solid precipitated, which was filtered and dried in vacuum to obtain the final product.

### Example 2: Synthesis of 4,6-dimethoxy-1,3,5-triazyn-2-yl stearate (10)

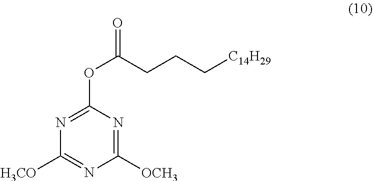

Compound (10) was synthesised from stearic acid and 2-chloro-4,6-dimethoxy-1,3,5-triazine following the general procedure described in Example 1.

¹H NMR (500 MHz, CDCl₃) δ 4.06 (s, 6H), 2.60 (t, J=7.5 Hz, 2H), 1.73 (m, 2H), 1.77-1.68 (m, 28H), 0.88 (t, J=6.9 Hz, 3H).

### Example 3: Synthesis of 6-methoxy-5-methyl-4-oxo-4,5-dihydro-1,3,5-triazyn-2-yl stearate (11)

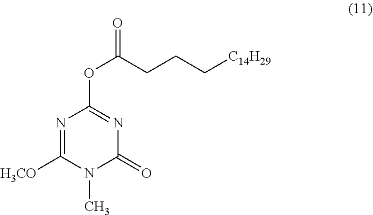

Compound (11) was synthesised from stearic acid and 2-chloro-4,6-dimethoxy-1,3,5-triazine following the general procedure described in Example 1.

¹H NMR (400 MHz, CDCl₃) δ 4.10 (s, 3H), 3.35 (s, 3H), 2.84 (t, J=7.4 Hz, 2H), 1.78-1.67 (m, 2H), 1.43-1.19 (m, 28H), 0.87 (t, J=6.7 Hz, 3H).

¹³C NMR (101 MHz, CDCl₃) δ 174.66, 160.13, 152.21, 148.83, 57.51, 40.30, 31.93, 29.70, 29.67, 29.66, 29.64, 29.58, 29.41, 29.36, 29.25, 28.81, 28.63, 23.61, 22.69, 14.11.

### Example 4: Synthesis of a Combination of 6-methoxy-5-methyl-4-oxo-4,5-dihydro-1,3,5-triazyn-2-yl stearate (11) and 4-methoxy-1-methyl-6-oxo-1,6-dihydro-1,3,5-triazin-2-yl stearate (13)

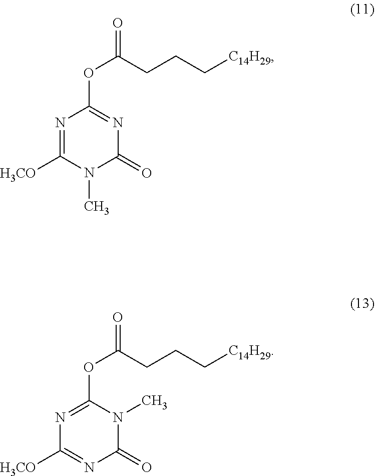

The combination of compounds (11) and (13) was synthesised from stearic acid and 2-chloro-4,6-dimethoxy-1,3,5-triazine following the general procedure described in Example 1. The amounts of compounds (11) and (13) may vary over time due to interconversion between the two isomeric structures.

¹H NMR (400 MHz, CDCl₃): δ 4.10 (s), 4.05 (s), 3.35 (s), 3.34 (s), 2.85 (m), 1.78-1.67 (m), 1.43-1.19 (m), 0.88 (m).

### Example 5: Synthesis of a Combination of 4,6-dimethoxy-1,3,5-triazyn-2-yl stearate (10) and 4,6-dimethoxy-1-stearoyl-1,3,5-triazin-2-one (12)

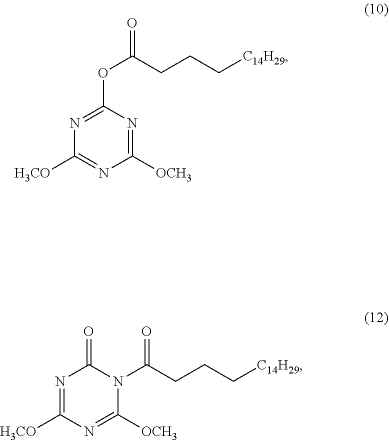

The combination of compounds (10) and (12) was synthesised from stearic acid and 2-chloro-4,6-dimethoxy-1,3,5-triazine following the general procedure described in Example 1. The amounts of compounds (10) and (12) may vary over time due to interconversion between the two isomeric structures.

¹H NMR (500 MHz, CDCl₃) δ 4.06 (s), 4.08 (s), 4.02 (s), 2.88 (t), 2.60 (t), 1.77-1.68 (m), 1.73 (m), 1.43-1.20 (m), 0.88 (t).

### Example 6: General Procedure for the Synthesis of Sphingolipids

A lysosphingolipid was dissolved in methanol, the triazine-based acylating agent of formula (10) (1 eq), or the triazine-based acylating agent of formula (11) (1 eq), or the combination of triazine-based acylating agents of formulas (11) and (13) (1 eq), or the combination of triazine-based acylating agents of formula (10) and (12) (1 eq), was added. The resulting suspension was stirred at about 50° C. until a TLC-analysis showed complete consumption of the starting material. Water was added, and the suspension was subsequently heated to reflux, and then cooled down the to a temperature between about 5° C. to about 10° C. A solid precipitated, which was filtered, washed with a methanol/water (5/1) mixture, and dried in vacuum to obtain the final product.

### Example 7: Synthesis of α-N-acetylneuraminosyl-(2-93)-O-D-galactopyranosyl-(1→4)—S-D-glucopyranosyl-(1→1′)—N-stearoyl-D-erythro-sphingosine (37), (GM3)

Compound (37) was synthesised from α-N-acetylneuraminosyl-(2→3)-O-β-D-galactopyranosyl-(1→4)-β-D-glucopyranosyl-(1→1′)-D-erythro-sphingosine and the triazine-based acylating agent of formula (10), or the triazine-based acylating agent of formula (11), or the combination of triazine-based acylating agents of formulas (11) and (13), or the combination of triazine-based acylating agents of formulas (10) and (12), following the general procedure described in Example 6.

MS spectrum: [M+H]⁺ 1181, [M+Na]⁺ 1203

### Example 8: Synthesis of β-D-galactopyranosyl-(1→4)-β-D-glucopyranosyl-(1→1′)—N-stearoyl-D-erythro-sphingosine (36)

Compound (36) was synthesised from β-D-galactopyranosyl-(1→4)-β-D-glucopyranosyl-(1→1′)-D-erythro-sphingosine and the triazine-based acylating agent of formula (10), or the triazine-based acylating agent of formula (11), or the combination of the triazine-based acylating agents of formulas (11) and (13), or the combination of the triazine-based acylating agents of formulas (10) and (12), following the general procedure described in Example 6.

MS spectrum: [M+H]⁺ 890, [M+Na]⁺ 913

### Example 9: Synthesis of β-D-glucopyranosyl-(1→1′)—N-stearoyl-D-erythro-sphingosine (34)

Compound (34) was synthesised from β-D-glucopyranosyl-(1→1′)-D-erythro-sphingosine and the triazine-based acylating agent of formula (10), or the triazine-based acylating agent of formula (11), or the combination of the triazine-based acylating agents of formulas (11) and (13), or the combination of the triazine-based acylating agents of formulas (10) and (12), following the general procedure described in Example 6.

MS spectrum: [M+H]⁺ 728, [M+Na]⁺ 750

1. A method for the production of a sphingolipid of formula (1): 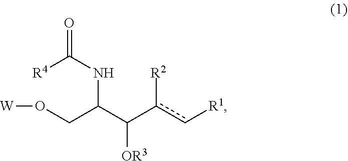
wherein
W is hydrogen or a glycosyl moiety,
R¹ is H, aryl, or a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of a hydroxyl group, an alkoxy group, an acyloxy group, an acylamido group, a thiol, a thioether or a phosphorus-containing functional group,
R² is H or —OR⁵, wherein R⁶ is selected from hydrogen, a substituted or unsubstituted C₁₋₆ alkyl, or a substituted or unsubstituted C₁₋₆ acyl,
the bond [symbol] may be a double or a single bond when R² is H, or is a single bond when R² is —OR⁵,
R³ is H, a substituted or unsubstituted C₁₋₆ alkyl, or a substituted or unsubstituted C₁₋₆ acyl,
R⁴ is selected from hydrogen, a substituted or unsubstituted aryl, a heteroalkyl, a substituted or unsubstituted C₁₋₃₁ alkyl, preferably a substituted or unsubstituted C₉₋₃₁ alkyl,
the method comprising:
reacting a lysosphingolipid of formula (2): 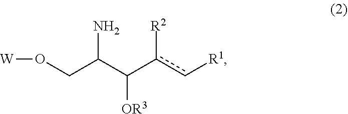
wherein W, the bond [symbol] , R¹, R² and R³ are as defined as for the sphingolipid of formula (1), with one or more triazine-based acylating agents of formula (3): 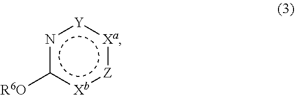
wherein 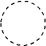
represents a conjugated system of bonds such that either two or three double bonds are present in the ring;
Y is selected from C(O—C(═O)R⁴), or C(═O),
X^{a} is selected from N, NR⁶, or N(C(═O)R⁴)
X^{b} is selected from N, or NR⁶
Z is selected from C(═O), or C(OR⁶),
and provided that:
when Y is C(O—C(═O)R⁴), Z is C(OR⁶), X^{a} and X^{b} are N, and three double bonds are present in the ring, or
when Y is C(O—C(═O)R⁴), Z is C(═O), one of X^{a} and X^{b} is N and the other group is NR⁶, and two double bonds are present in the ring, or
when Y is C(═O), Z is C(OR⁶), X^{a} is N(C(═O)R⁴), X^{b} is N, and two double bonds are present in the ring,
and wherein
R⁴ is as defined as for the sphingolipid of formula (1),
R⁶ is selected from methyl, ethyl, 2,2,2-trifluoroethyl, and substituted or unsubstituted benzyl, thereby producing the sphingolipid of formula (1).

2. The method according to claim 1, wherein the triazine-based acylating agent of formula (3) is a triazine-based acylating agents of formulas (4), or (5): 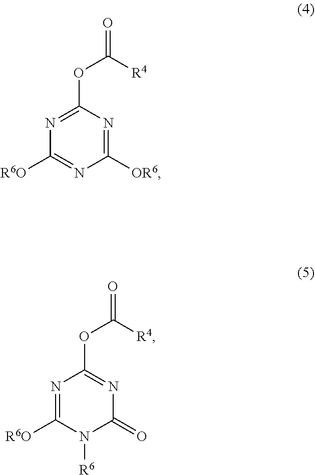
wherein R⁴ and R⁶ are as defined as for the triazine-based acylating agent of formula (3).

3. The method according to claim 1, wherein the triazine-based acylating agent of formula (3) is a combination comprising triazine-based acylating agents of formulas (4), (5), (6) and (7): 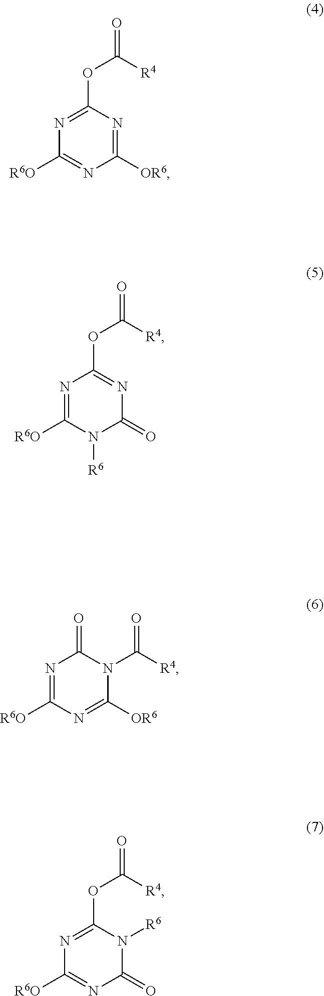
wherein
R⁴ and R⁶ are as defined as for the acylating agent of formula (3), and
wherein each of said compounds is present, in said combination, in the amount from about 1% to about 99%.

4. The method according to any one of claims 1 to 3, wherein R⁴ of the sphingolipid of formula (1), and R⁴ of the triazine-based acylating agent of formulas (3), (4), (5), (6), and (7) is a substituted or unsubstituted C₉₋₃₁ alkyl selected from the group consisting of substituted or unsubstituted C₉₋₃₁ alkyls of formula (8) and (9): 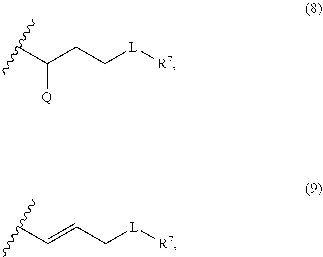
wherein
Q is selected from —H, —OH, —Cl, —Br, or —I,
L is a straight-chain C₆₋₂₈ alkylene, which may be saturated or contain one or more double bonds and or triple bonds,
R⁷ is hydrogen, or —OR⁸, wherein R⁸ is hydrogen or a straight-chain C₂₋₃₀ acyl which may be saturated or contain one or more double bonds.

5. The method according to claim 4 wherein L, of the substituted or unsubstituted C₉₋₃₁ alkyl of formula (8) or (9), is selected from a straight-chain saturated C₁₄ alkylene, straight-chain saturated or unsaturated C₁₆ alkylene, or a straight-chain saturated or unsaturated C₂₆₋₂₈ alkylene.

6. The method according to claims 4 or 5, wherein for the substituted or unsubstituted C₉₋₃₁ alkyl of formula (8) or (9), Q is selected from —H, or —OH, L is a straight-chain saturated C₁₄ alkylene, and R⁷ is hydrogen.

7. The method according to any one of claims 1 to 6, wherein R⁶ of the triazine-based acylating agent of formulas (3), (4), (5), (6) and (7) is methyl.

8. The method according to any one of claims 2 to 7, wherein the triazine-based acylating agent of formula (4), (5), (6) and (7) are triazine based acylating agents of formulas (10), (11), (12), and (13), respectively:

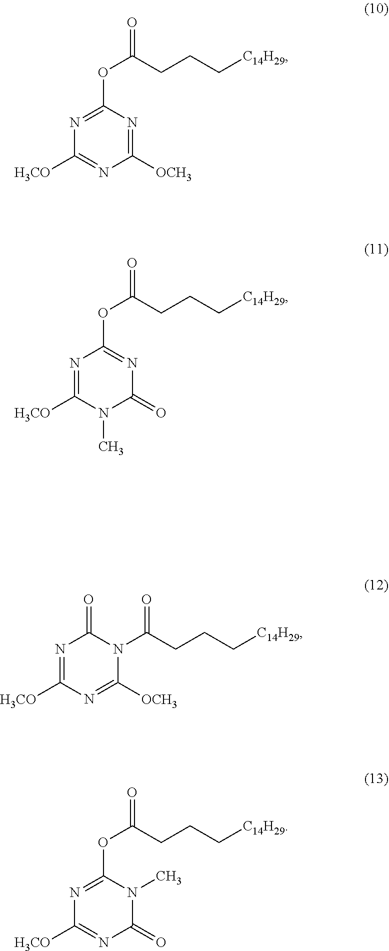

9. The method according to any one of claims 1 to 8, wherein the lysosphingolipid of formula (2) is in the form of a salt.

10. The method according to claim 9, wherein the salt is a hydrochloride, a hydrobromide, a sulphate, a phosphate, a polyphosphate, an acetate, a p-toluene sulfate, a methane sulfate, a trifluoromethanesulfate, or a perchlorate salt of the lysosphingolipid of formula (2), preferably a hydrochloride salt of the lysosphingolipid of formula (2).

11. The method according to any one of claims 1 to 10, further comprising the use of a base.

12. The method according to claim 11, wherein the base is selected from NaOH, KOH, LiOH, Ca(OH)₂, triethylamine, N,N-diisopropylethylamine, and pyridine.

13. The method according to any one of claims 1 to 12, further comprising the use of a solvent.

14. The method according to claim 13, wherein the solvent is a polar solvent, or a mixture of one or more polar solvents, and wherein the polar solvent is preferably selected from water, methanol, ethanol, propanol, isopropanol, butanol, isobutanol.

15. The method according to any one of claims 1 to 14, wherein the lysosphingolipid of formula (2) is a lysosphingolipid of formula (14) or formula (15): 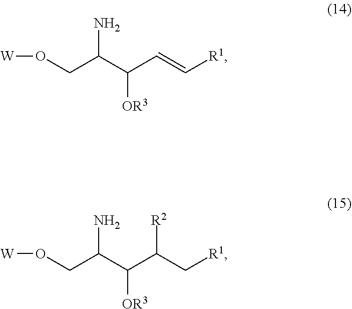
wherein
W, R¹, R², and R³, are as defined as for the lysosphingolipid of formula (2).

16. The method according to claim 15, wherein the stereochemical configuration of the C-2, C-3, and C-4 carbon atoms of the lysosphingolipid of formula (14) is (2S,3R,4E), and wherein the stereochemical configuration of the C-2, C-3, and C-4 carbon atoms of the lysosphingolipid of formula (15) is (2S,3S,4R).

17. The method according to claims 15 or 16, wherein for the lysosphingolipid of formulas (14), or (15), R¹ is a substituted or unsubstituted C₁₃ alkyl, R² is hydrogen or —OH, and R³ is hydrogen.

18. The method according to any one of claims 1 to 17, wherein W of the sphingolipid of formula (1), and W of the lysosphingolipid of formula (2), (14), or (15) is a glycosyl moiety, and wherein the glycosyl moiety is selected from Glc1-, Gal1-, Galβ1-4Glc1-.

19. The method according to any one of claims 1 to 17, wherein W of the sphingolipid of formula (1), and W of the lysosphingolipid of formula (2), (14), or (15) is a glycosyl moiety, and wherein the glycosyl moiety is the oligosaccharide portion of a ganglioside selected from GM1a, GM1b, GD1a, GD1b, GD3, GT1b, GT3, GQ1b, GM3, and GM4.

20. The method according to any one of claims 1 to 17, wherein W of the sphingolipid of formula (1), and W of the lysosphingolipid of formula (2), (14), or (15) is a glycosyl moiety, and wherein the glycosyl moiety is that of a human milk oligosaccharide.

21. The method according to any one of claims 1 to 17, wherein W of the sphingolipid of formulas (1), and W of the lysosphingolipid of formula (2), (14), or (15) is hydrogen.

22. The method according to any one of claims 1 to 21, wherein the method further comprising a step of isolating the sphingolipid of formula (1).

23. The method according to claim 22, wherein the sphingolipid of formula (1) is isolated via precipitation.

24. The method according to anyone of claims 1 to 23, wherein the method further comprising steps of producing the triazine-based acylating agent formula (3), or the combination thereof.

25. The method according to claim 24, wherein the triazine-based acylating agent of formula (3), or the combination thereof, is produced via the steps of:
reacting a carboxylic acid of formula (16): 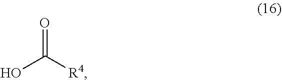
wherein
R⁴ is as defined as for the sphingolipid of formula (1),
with a compound of formula (17): 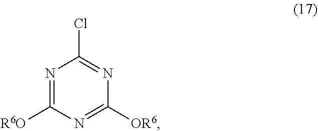
wherein R⁶ is as defined as for the triazine-based acylating agent of formula (3), or the combination thereof,
in the presence of an organic base, thereby producing the triazine-based acylating agents of formula (3), or the combination thereof;
isolating the triazine-based acylating agent of formula (3), or the combination thereof, via precipitation;
and wherein the steps of producing the triazine-based acylating agent of formula (3), or the combination thereof, are performed in a non-halogenated solvent.

26. The method according to claim 25, wherein the steps producing the triazine-based acylating agent of formula (3), or the combination thereof, are performed in the same non-halogenated solvent

27. The method according to claims 25 or 26, wherein the non-halogenated solvent is a ketone selected from acetone, diethyl ketone, methyl isobutyl ketone, or butan-2-one, preferably acetone.

28. The method according to claims 25 or 26, wherein the non-halogenated solvent is an alcohol selected from methanol, ethanol, propanol, isopropanol, butanol, isobutanol, preferably methanol.

29. The method according to claims 25 or 26, wherein the non-halogenated solvent is an aliphatic hydrocarbon selected from petroleum ether, hexane, or an isomeric mixture thereof, n-heptane, or an isomeric mixture thereof, octane, or an isomeric mixture thereof.

30. The method according to any one of claims 25 or 26, wherein the non-halogenated solvent is a mixture of two alcohols.

31. The method according to claim 30, wherein the mixture of two alcohols is selected from a mixture of methanol and ethanol, methanol and propanol, methanol and isopropanol, methanol and butanol, or methanol and isobutanol, preferably a mixture of methanol and ethanol.

32. The method according to claim 27, further comprising the addition of water to the step of isolating the triazine-based acylating agent of formula (3).

33. The method according to any one of claims 25 to 32, wherein the step of reacting a carboxylic acid of formula (16) with the compound of formula (17) is performed at a temperature between about 30° C. and about 100° C., preferably between about 30° C. and about 55° C.

34. The method according to any one of claims 25 to 33, wherein the step of isolating the triazine-based acylating agent of formula (3), or the combination thereof, is performed at a temperature between about −20° C. and about 25° C., preferably at a temperature between about −10° C. and about 25° C., even more preferably at a temperature between about 5° C. and about 25° C.

35. The method according to any one of claims 25 to 34, wherein the organic base is selected from 4-methylmorpholine, 1,4-diazabicyclo[2.2.2]octane, preferably 4-methylmorpholine.

36. A triazine based acylating agent of formula (3), or a combination thereof: 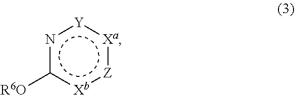
wherein 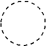
represents a conjugated system of bonds such that either two or three double bonds are present in the ring;
Y is selected from C(O—C(═O)R⁴), or C(═O),
X^{a} is selected from N, NR⁶, or N(C(═O)R⁴)
X^{b} is selected from N, or NR⁶
Z is selected from C(═O), or C(OR⁶),
provided that:
when Y is C(O—C(═O)R⁴), Z is C(OR⁶), X^{a} and X^{b} are N, and three double bonds are present in the ring, or
when Y is C(O—C(═O)R⁴), Z is C(═O), one of X^{a} and X^{b} is N and the other group is NR⁶, and two double bonds are present in the ring, or
when Y is C(═O), Z is C(OR⁶), X^{a} is N(C(═O)R⁴), X^{b} is N, and two double bonds are present in the ring,
and wherein
R⁶ is selected from methyl, ethyl, 2,2,2-trifluoroethyl, and substituted or unsubstituted benzyl,
R⁴ is a substituted or unsubstituted C₉₋₃₁ alkyl selected from the group consisting of substituted or unsubstituted C₉₋₃₁ alkyls of formula (8) and (9): 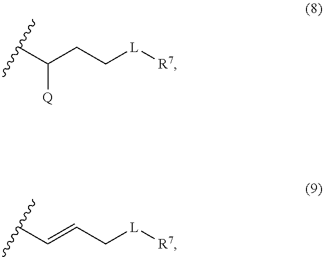
wherein
Q is selected from —H, —OH, —Cl, —Br, or —I,
L is a straight-chain C₆₋₂₈ alkylene, which may be saturated or contain one or more double bonds and or triple bonds,
R⁷ is hydrogen, or —OR⁸, wherein R⁸ is hydrogen or a straight-chain C₂₋₃₀ acyl which may be saturated or contain one or more double bonds;
and provided that:
when R⁶ is a methyl and Q and R⁷ are hydrogen, L of the alkyl group of formula (8) is not an unsaturated straight-chain C₁₄ alkylene, or L of the alkyl group of formula (8) is not a straight-chain C₁₅ alkylene, or L of the alkyl group of formula (8) is not a straight-chain C₁₈ alkylene.

37. The triazine-based acylating agent according to claim 36, wherein L is selected from a straight-chain saturated C₁₄ alkylene, straight-chain saturated or unsaturated C₁₆ alkylene, or a straight-chain saturated or unsaturated C₂₆₋₂₈ alkylene.

38. The triazine-based acylating agent according to claims 36 or 37, wherein R⁶ is methyl, Q is selected from —H, or —OH, L is a straight-chain saturated C₁₄ alkylene, and R⁷ is hydrogen.

39. The triazine-based acylating agent according to any one of claims 36 to 38, wherein the triazine-based acylating agent of formula (3) is a triazine-based acylating agent selected from the group consisting of triazine-based acylating agents of formula of formula (10) and formula (11):

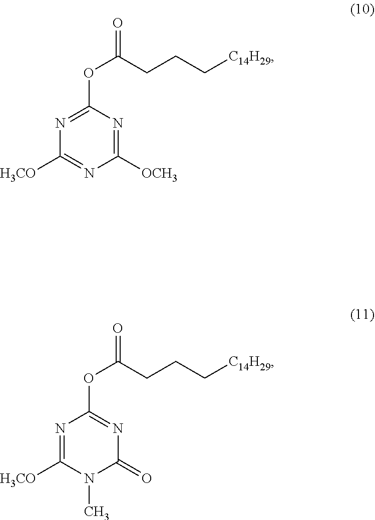

40. A combination comprising triazine base acylating agents of formula (10)-(13): 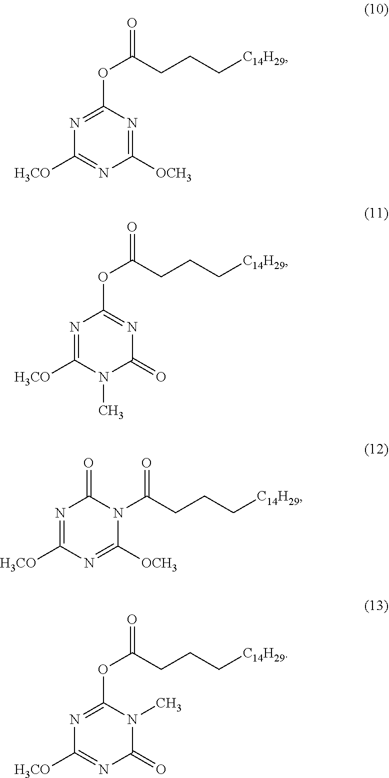
and wherein each of said triazine is present, in said combination, in the amount from about 1% to about 99%.

41. The combination according to claim 40, wherein the combination comprising triazine base acylating agents of formula (10) and (12), and wherein each of said triazine is present, in said combination, in the amount from about 1% to about 99%.

42. The combination according to claim 40, wherein the combination comprising triazine base acylating agents of formula (11) and (13), and wherein each of said triazine is present, in said combination, in the amount from about 1% to about 99%.
